(19) United States
(12) Patent Application Publication
(10) Pub. No.: US 2022/0370561 A1
(43) Pub. Date: Nov. 24, 2022

(54) LITHIUM AS A MONOTHERAPY AND/OR STEM CELL ADJUVANT THERAPY FOR PULMONARY FIBROSIS

(71) Applicant: Therapeutic Solutions International, Inc., Oceanside, CA (US)

(72) Inventors: Thomas E. Ichim, Oceanside, CA (US); Timothy G. Dixon, Oceanside, CA (US); Wals Kaihani, Oceanside, CA (US); Famela Ramos, Oceanside, CA (US); James Veltmeyer, Oceanside, CA (US)

(73) Assignee: Therapeutic Solutions International, Inc., Oceanside, CA (US)

(21) Appl. No.: 17/748,943

(22) Filed: May 19, 2022

Related U.S. Application Data
(60) Provisional application No. 63/191,770, filed on May 21, 2021.

(51) Int. Cl. A61K 38/19; A61K 33/24; A61K 35/28; A61K 31/395

(52) U.S. Cl. CPC A61K 38/193; A61K 33/24; A61K 35/28; A61K 31/395; A61K 38/19

(57) ABSTRACT

Disclosed are compositions of matter, therapeutics, and protocols useful for reduction and/or reversion of pulmonary fibrosis. In one specific embodiment lithium chloride is administered together with a regenerative cell in a patient suffering from, or at risk of pulmonary fibrosis. In one embodiment said lithium chloride is administered as an adjuvant to a regenerative therapy, wherein said regenerative therapy is a gene therapy, a protein therapy, a cell therapy, or a tissue transplant. In one embodiment lithium chloride, or a salt thereof is utilized alone, or with a regenerative means, to evoke preservation and/or elongation of telomere length in pulmonary tissue.

### CROSS-REFERENCE TO RELATED APPLICATIONS

This application claims the benefit of priority to U.S. Provisional Application No. 63/191,770 filed May 21, 2021, the entire contents of which is incorporated herein by reference.

### FIELD OF THE INVENTION

The invention pertains to the treatment, prevention, or amelioration of pulmonary fibrosis, using lithium as a therapeutic agent whether by itself or as a regenerative cell adjuvant.

### BACKGROUND

It is recognized that coronaviruses (CoVs) are a single stranded positive sense RNA viruses which include four genera (alpha, beta, delta, and gamma) [1]. Infectivity of CoVs is mediated by the envelope spike (S) glycoprotein which binds to its cellular receptors angiotensin-converting enzyme 2 (ACE2) and dipeptidyl peptidase 4 (DPP4) for SARS-CoV and MERS-CoV, respectively [2, 3]. In the case of the novel COVID-19 virus, it is over 99% similar to SAR-CoV-2 which is a new type of beta genera. This is based on 10 sequenced samples collected from the original location of the outbreak [4]. SAR-2-CoV preferentially infects the type 2 pulmonary epithelial cells, in the lungs, which express ACE2 [5]. Immune responses to the family of coronaviruses are associated with induction of type 1 interferons, especially interferon beta, which originally was termed “MSC interferon”. Clinical trials are ongoing assessing intranasal interferon beta (https://pharmaphorum.com/news/synairgen-doses-first-patients-with-covid-19-drug/) for COVID-19. Interferons not only induce expression of genes that block viral replication, but also are responsible for stimulating natural killer (NK) cells, which selectively kill virally infected cells. Interestingly, exogenous allogeneic NK cells have been recently cleared by FDA for treatment of COVID-19 (https://techcrunch.com/2020/04/02/venture-backed-celularity-receives-fda-approval-for-early-trials-of-a-new-cell-therapy-for-covid-19/). One ideal treatment for prevention of SARS-CoV-2 progression would be an agent which induces interferon production in the lung.

Mortality from COVID-19 is caused by acute respiratory distress syndrome (ARDS) [6, 7], which is caused by unrestrained cytokine release, also known as “cytokine storm”, and is characterized by fluid leakage, diffuse inflammation, and disseminated intravascular coagulation, all of which cause impaired alveolar gas exchange. Approximately 35-45% of patients with ARDS will die [8].

The feasibility of utilizing cell based approaches to ARDS has been demonstrated in animal models [9-11], in which researchers have shown reduction of pulmonary injury, water leakage, and neutrophil accumulation. Furthermore, analysis of 342 systemic infusions and 57 bronchial instillations (204 recipients) of cells of various origins for ARDS and other pulmonary issues demonstrated safety in early clinical trials [12, 13].

Unfortunately, to date, no treatments appear effective for Post-COVID associated pulmonary fibrosis.

### SUMMARY

Preferred embodiments are directed to methods of treating an individual diagnosed with, suspected of having, or preventing in an individual at risk for developing, pulmonary fibrosis comprising administration of a therapeutically effective amount of a lithium salt alone or together with a regenerative means.

Preferred methods include embodiments wherein said regenerative means are mesenchymal stem cells, or stem cell-like cells derived from a source of tissues selected from a group comprising of: a) dermal; b) placental; c) hair follicle; d) deciduous tooth; e) omentum; f) placenta; g) Wharton's jelly; h) bone marrow; i) adipose tissue; j) amniotic membrane; k) amniotic fluid; l) peripheral blood.

Preferred methods include embodiments wherein said peripheral blood is mobilized to enhance concentration of stem cells

Preferred methods include embodiments wherein said mobilization is achieved by treatment of said patient with an agent selected from a group comprising of: a) G-CSF; b) M-CSF; c) GM-CSF; d) Mozibil; and e) flt-3 ligand.

Preferred methods include embodiments wherein said stem cell is either: a) allogeneic; b) autologous; or c) xenogeneic to the recipient.

Preferred methods include embodiments wherein said pulmonary fibrobis is caused by factors selected from a group comprising of: a) cytokine storm; b) immunological cell infiltration; c) bacterial infection; d) viral infection; e) systemic inflammatory response syndrome; f) systemic inflammation; g) acute radiation syndrome; and h) sepsis.

Preferred methods include embodiments, wherein said stem cells are administered intravenously.

Preferred methods include embodiments wherein said stem cells are administered intranasally.

Preferred methods include embodiments wherein said stem cells are administered intratracheally.

Preferred methods include embodiments wherein said stem cells are pre-activated with an agent capable of enhancing MSC therapeutic activity.

Preferred methods include embodiments wherein said stem cells therapeutic activity is selected from a group comprising of: a) mobility towards a chemotactic agent; b) production of anti-inflammatory agents; and c) production of anti-apoptotic agents.

Preferred methods include embodiments wherein said mobility towards a chemotactic agent is mediated by enhanced expression of a receptor associated with enhanced chemotaxis.

Preferred methods include embodiments wherein said receptor associated with enhanced chemotaxis is CXCR4.

Preferred methods include embodiments wherein said anti-inflammatory factors are selected from a group comprising of: a) IL-4; b) IL-10; c) IL-13; d) IL-20; e) IL-27; f) IL-35; g) PGE-2; h) indolamine 2,3 deoxygenase; i) TGF-beta; and J) EGF.

Preferred methods include embodiments wherein said stem cells are modified to express enhanced levels of therapeutic cytokines.

Preferred methods include embodiments wherein said therapeutic cytokines are selected from a group comprising of: a) cytokines that inhibit apoptosis; b) cytokines that act as growth factors; and c) cytokines which act as immune modulators/anti-inflammatory agents.

Preferred methods include embodiments wherein said cytokines that inhibit apoptosis are selected from a group comprising of: a) EGF; b) VEGF; and c) angiopoietin.

Preferred methods include embodiments wherein said cytokines that act as growth factors are selected from a group comprising of: a) HGF; b) FGF-1; c) FGF-2; d) KGF; and e) CTNF.

Preferred methods include embodiments wherein said cytokines which act as immune modulators/anti-inflammatory agents are selected from a group comprising of: a) IL-4; b) IL-10; c) IL-13; d) IL-20; e) IL-27; f) IL-35; g) PGE-2; h) indolamine 2,3 deoxygenase; i) TGF-beta; and J) neuroaminidase.

Preferred embodiments include methods of reprogramming monocytes in the lung of a patient suffering or having the potential to suffer from ARDS comprising administration of a therapeutically effective amount of stem cells alone or in combination with a lithium salt.

Preferred methods include embodiments wherein said stem cells are derived from a source of tissues selected from a group comprising of: a) dermal; b) placental; c) hair follicle; d) deciduous tooth; e) omentum; f) placenta; g) Wharton's jelly; h) bone marrow; i) adipose tissue; j) amniotic membrane; k) amniotic fluid; l) peripheral blood.

Preferred methods include embodiments wherein said peripheral blood is mobilized to enhance concentration of stem cells before isolation of stem cells.

Preferred methods include embodiments wherein said mobilization is achieved by treatment of said patient with an agent selected from a group comprising of: a) G-CSF; b) M-CSF; c) GM-CSF; d) Mozibil; and e) flt-3 ligand.

Preferred methods include embodiments wherein said stem cells is either: a) allogeneic; b) autologous; or c) xenogeneic to the recipient.

Preferred methods include embodiments wherein said pulmonary fibrosis is caused by factors selected from a group comprising of: a) cytokine storm; b) immunological cell infiltration; c) bacterial infection; d) viral infection; e) systemic inflammatory response syndrome; f) systemic inflammation; g) acute radiation syndrome; and h) sepsis.

Preferred methods include embodiments wherein said stem cells are administered intravenously.

Preferred methods include embodiments wherein said stem cells are administered intranasally.

Preferred methods include embodiments wherein said stem cells are administered intratracheally.

Preferred methods include embodiments wherein said stem cells are pre-activated with an agent capable of enhancing MSC therapeutic activity.

Preferred methods include embodiments wherein said stem cells therapeutic activity is selected from a group comprising of: a) mobility towards a chemotactic agent; b) production of anti-inflammatory agents; and c) production of anti-apoptotic agents.

Preferred methods include embodiments wherein said mobility towards a chemotactic agent is mediated by enhanced expression of a receptor associated with enhanced chemotaxis.

Preferred methods include embodiments wherein said receptor associated with enhanced chemotaxis is CXCR4.

Preferred methods include embodiments wherein said anti-inflammatory factors are selected from a group comprising of: a) IL-4; b) IL-10; c) IL-13; d) IL-20; e) IL-27; f) IL-35; g) PGE-2; h) indolamine 2,3 deoxygenase; i) TGF-beta; and J) EGF.

Preferred methods include embodiments wherein said MSC are modified to express enhanced levels of therapeutic cytokines.

Preferred methods include embodiments wherein said therapeutic cytokines are selected from a group comprising of: a) cytokines that inhibit apoptosis; b) cytokines that act as growth factors; and c) cytokines which act as immune modulators/anti-inflammatory agents.

Preferred methods include embodiments wherein said cytokines that inhibit apoptosis are selected from a group comprising of: a) EGF; b) VEGF; and c) angiopoietin.

Preferred methods include embodiments wherein said cytokines that act as growth factors are selected from a group comprising of: a) HGF; b) FGF-1; c) FGF-2; d) KGF; and e) CTNF.

Preferred methods include embodiments wherein said cytokines which act as immune modulators/anti-inflammatory agents are selected from a group comprising of: a) IL-4; b) IL-10; c) IL-13; d) IL-20; e) IL-27; f) IL-35; g) PGE-2; h) indolamine 2,3 deoxygenase; i) TGF-beta; and J) neuroaminidase.

Preferred methods include embodiments wherein administration of a second therapeutic treatment is performed, wherein the second therapeutic treatment is administered sequentially or simultaneously.

Preferred methods include embodiments wherein the second therapeutic treatment is ventilation, a glucocorticoid, a surfactant, inhaled nitric oxide, an antioxidant, a protease inhibitor, a recombinant human activated protein C, a .beta.2-agonist, lisofylline, a statin, inhaled heparin, a diuretic, a sedative, an analgesic, a muscle relaxant, an antibiotic, inhaled prostacyclin, inhaled synthetic prostacydin analog, ketoconazole, alprostadil, keratinocyte growth factor, beta-agonists, human monoclonal antibody (mAb) against tissue factor VIIa (TS factor 7a), interferon receptor agonists, insulin, perfluorocarbon, budesonide, recombinant human angiotensin-converting enzyme (ACE), recombinant human Clara cell 10 kDa (CC10) protein, tissue plasminogen activator, human mesenchymal stem cells, or nutritional therapy.

Preferred methods include embodiments wherein the second therapeutic treatment is methylprednisolone, dexamethasone, prednisone, prednisolone, betamethasone, triamcinolone, triamcinolone acetonide, beclometasone, albuterol, lisofylline, rosuvastatin, inhaled heparin, inhaled nitric oxide, recombinant human activated protein C, ibuprofen, naproxen, acetaminophen, cisatracurium besylate, procysteine, acetylcysteine, inhaled prostacydin, ketoconazole, alprostadil, keratinocyte growth factor, beta-agonists, human monoclonal antibody (mAb) against tissue factor VIIa (TS factor 7a), insulin, perfluorocarbon, budesonide, recombinant human angiotensin-converting enzyme (ACE), recombinant human Clara cell 10 kDa (CC10) protein, tissue plasminogen activator, human mesenchymal stem cells, a nutritional therapy, a combination of omega-3 fatty acids, antioxidants, .gamma.-linolenic acids with isocaloric foods, or mechanical ventilation.

Preferred methods include embodiments wherein said stem cells are endowed with ability to suppress viral infection.

Preferred methods include embodiments wherein said suppression of viral infection is achieved through production of interferon.

Preferred methods include embodiments wherein said interferon is selected from a group comprising of: a) interferon alpha; b) interferon beta; c) interferon gamma; d) interferon tau; and e) interferon omega.

Preferred methods include embodiments wherein said ability to suppress viral infection is accomplished by treatment of stem cells with an activator of toll like receptor.

Preferred methods include embodiments wherein said toll like receptor is TLR-1.

Preferred methods include embodiments wherein said activator of TLR-1 is Pam3CSK4.

Preferred methods include embodiments wherein said toll like receptor is TLR-2.

Preferred methods include embodiments wherein said activator of TLR-2 is HKLM.

Preferred methods include embodiments wherein said toll like receptor is TLR-3.

Preferred methods include embodiments wherein said activator of TLR-3 is Poly:IC.

Preferred methods include embodiments wherein said toll like receptor is TLR-4.

Preferred methods include embodiments wherein said activator of TLR-4 is LPS.

Preferred methods include embodiments wherein said activator of TLR-4 is Buprenorphine.

Preferred methods include embodiments wherein said activator of TLR-4 is Carbamazepine.

Preferred methods include embodiments wherein said activator of TLR-4 is Fentanyl.

Preferred methods include embodiments wherein said activator of TLR-4 is Levorphanol.

Preferred methods include embodiments wherein said activator of TLR-4 is Methadone.

Preferred methods include embodiments wherein said activator of TLR-4 is Cocaine.

Preferred methods include embodiments wherein said activator of TLR-4 is Morphine.

Preferred methods include embodiments wherein said activator of TLR-4 is Oxcarbazepine.

Preferred methods include embodiments wherein said activator of TLR-4 is Oxycodone.

Preferred methods include embodiments wherein said activator of TLR-4 is Pethidine.

Preferred methods include embodiments wherein said activator of TLR-4 is Glucuronoxylomannan from Cryptococcus.

Preferred methods include embodiments wherein said activator of TLR-4 is Morphine-3-glucuronide.

Preferred methods include embodiments wherein said activator of TLR-4 is lipoteichoic acid.

Preferred methods include embodiments wherein said activator of TLR-4 is β-defensin 2.

Preferred methods include embodiments wherein said activator of TLR-4 is small molecular weight hyaluronic acid.

Preferred methods include embodiments wherein said activator of TLR-4 is fibronectin EDA.

Preferred methods include embodiments wherein said activator of TLR-4 is snapin.

Preferred methods include embodiments wherein said activator of TLR-4 is tenascin C.

Preferred methods include embodiments wherein said toll like receptor is TLR-5.

Preferred methods include embodiments wherein said activator of TLR-5 is flagellin.

Preferred methods include embodiments wherein said toll like receptor is TLR-6.

Preferred methods include embodiments wherein said activator of TLR-6 is FSL-1.

Preferred methods include embodiments wherein said toll like receptor is TLR-7.

Preferred methods include embodiments wherein said activator of TLR-7 is imiquimod.

Preferred methods include embodiments wherein said toll like receptor of TLR-8.

Preferred methods include embodiments wherein said activator of TLR8 is ssRNA40/LyoVec.

Preferred methods include embodiments wherein said toll like receptor of TLR-9.

Preferred methods include embodiments wherein said activator of TLR-9 is a CpG oligonucleotide.

Preferred methods include embodiments wherein said activator of TLR-9 is ODN2006.

Preferred methods include embodiments wherein said activator of TLR-9 is Agatolimod.

### BRIEF DESCRIPTION OF THE DRAWINGS

FIG. 1 is a bar graph showing that lithium enhances the ability of JadiCells to increase T regulatory cell numbers in mice.

FIG. 2 is a bar graph showing JadiCells are superior to BM and Adipose MSCs in enhancing antifibrotic activity.

FIG. 3 is a bar graph showing lithium chloride enhances antifibrotic activities of JadiCells

### DESCRIPTION OF THE INVENTION

The invention discloses the novel, useful, and unexpected finding that administration of regenerative cells, particularly, stem cells, more particularly, mesenchymal stem cells together with a lithium salt, induces a protective effect in pulmonary fibrosis. In some embodiment of the invention, transfer of MSC possessing a younger biological age than the recipient is disclosed for the purposes of reducing fibrosis, and/or preserving, and/or increasing telomere length. More specifically, the invention provides means of using MSC together with a lithium salt, and/or products derived from said cells to inhibit the rate of telomere shortening, stabilize telomere length, as well as elongate telomere length. In one embodiment the invention provides the administration of MSC together with lithium salts, or lithium chloride, cells as a means of enhancing telomere length. Enhancement of telomere length may be performed in situations where inhibition of senescence may be desired, as in the case of aging, or particularly in the case of pulmonary aging associated with Post-COVID lung pathology. In another embodiment the invention provides means of treating conditions associated with acceleration of telomere reduction such as idiopathic pulmonary fibrosis, in which telomere reduction has been well documented [14-22].

Telomerase is a ribonucleoprotein responsible for regulating proliferative potential and preventing senescence in tumorigenic, germline and immortalized cells. It is composed of two essential subunits, among others. In humans, these subunits include: hTERC, which contains a telomeric RNA template that the catalytic reverse transcriptase subunit hTERT reads. Together these subunits operate to extend telomeres in a 3′-5′ direction (Nakamura and Cech, 1998). hTERT is the limiting factor for telomerase activity. This is due to its lower expression levels compared to hTERC, and thus, serves as the major regulator for telomerase activity. This said, telomerase fulfils its core function within the nucleus, where it elongates and maintains telomere length. This maintenance allows not only the continuation of normal cellular processes, but also improves the overall proliferative potential of cells.

Aside from telomere extension, telomerase/hTERT has extra-telomeric functions other than telomere maintenance. One such function is that hTERT may aid in regulating gene expression; especially genes of the Wnt and Myc pathway responsible for cell cycle initiation and proliferation/Furthermore, telomerase has been shown to play a role in mitochondrial protection against oxidative stress. This protective function is conveyed when cells are introduced to hypoxic stress, which causes the migration of nuclear hTERT to the mitochondria. Interestingly, aside from mitochondrial protection, hTERT may also play a role in mtDNA replication and repair. Thus, the additional functions that hTERT/telomerase has been found to display, further highlight its vital presence in regulating and promoting cell viability. The chromosomal ends that telomerase extends, known as telomeres, are composed of genomic TTAGGG repeats and associated protein structures. These repeats and related proteins together “cap” linear eukaryotic chromosomes for protection. A few of these telomere-related proteins include TRF1, TRF 2 and POT1. These proteins interact with the telomeres and each other to form the “shelterin” complex. This complex facilitates the folding of telomeres to form a telomere loop (t-loop) to prevent telomere degradation). This said, telomeres and their related proteins fulfil a vital function in protecting against genomic DNA damage. In addition, these telomeric structures also aid to distinguish between normal chromosomes and double stranded breaks. This damage is caused by the imperfect replicating nature of DNA polymerase; that generates gaps during lagging strand synthesis Additionally, DNA synthesis follows a unidirectional path (3′ to 5′), resulting in DNA ends not being fully replicated, otherwise known as the “end replication problem” (Harley et al., 1990). Thus, telomeres as well as their extension prevent the of genetic information, ensuring genomic stability and in turn cell viability.

Telomeres are known to be involved in a number of diseases and/or medical conditions. The telomere related diseases and/or telomere related medical conditions may be, for example, at least one of, but not limited to, the following group: dyskeratosis congenital, cancer, cellular ageing (cellular senescence), idiopathic pulmonary fibrosis, Hoyeraal-Hreiderasson syndrome, Hutchinson-Gilford progeria, aplastic anemia and age-related diseases. Particularly relevant, owing to their great impact on the lives of humans, is cancer, cellular ageing (senescence) and age-related diseases. Age-related diseases may include osteoporosis, type II diabetes, atherosclerosis and cardiovascular disease. In other embodiments of the invention, MSC cell administration is used to increase telomere length in conditions associated with telomere shortening. For example, studies have shown correlations between shortening of leukocyte telomeres and cardiac conditions. In one study, Haver et al. assessed leucocyte telomere length in 3275 patients with chronic ischaemic systolic heart failure participating in the COntrolled ROsuvastatin multiNAtional Trial in Heart Failure (CORONA) study. The primary composite endpoint was cardiovascular death, non-fatal myocardial infarction, and non-fatal stroke, which occurred in 575 patients during follow-up. They observed a significant association of leucocyte telomere lengths with the primary endpoint (hazard ratio 1.10; 95% confidence interval 1.01-1.20; P=0.03) [23]. One therapeutic cell type for the target of the invention is the endothelial progenitor cell (EPC), whose activity has been demonstrated to correlate with superior cardiovascular health [24-26]. Indeed telomere length has been shown to correlate with EPC activity [27-30]. It is known that numerous factors contribute to telomere shortening in addition to aging, these include DNA damage, inflammation, and oxidative stress. Both cardiovascular risk factors and common cardiovascular diseases, such as atherosclerosis, heart failure, and hypertension, are associated with short leucocyte telomeres [31],

For the practice of the invention, a preferred embodiment is the administration of MSC together with lithium salts or chloride intravenously at concentrations sufficient to increase telomere length. MSC cells in some embodiments refers to cells that are (1) adherent to plastic, (2) express CD73, CD90, and CD105 antigens, while being CD14, CD34, CD45, and HLA-DR negative, and (3) possess ability to differentiate to osteogenic, chondrogenic and adipogenic lineage.

MSC can be derived from any tissue including, but not limited to, bone marrow, adipose tissue, amniotic fluid, endometrium, trophoblast-derived tissues, cord blood, Wharton jelly, placenta, amniotic tissue, derived from pluripotent stem cells, and tooth. In some definitions of “MSCs”, said cells include cells that are CD34 positive upon initial isolation from tissue but are similar to cells described about phenotypically and functionally. As used herein, “MSC” may include cells that are isolated from tissues using cell surface markers selected from the list comprised of NGF-R, PDGF-R, EGF-R, IGF-R, CD29, CD49a, CD56, CD63, CD73, CD105, CD106, CD140b, CD146, CD271, MSCA-1, SSEA4, STRO-1 and STRO-3 or any combination thereof.

Said MSC may be expanded and utilized by administration themselves, or may be cultured in a growth media in order to obtain conditioned media, the term Growth Medium generally refers to a medium sufficient for the culturing of umbilicus-derived MSC cells. In particular, one presently preferred medium for the culturing of the cells of the invention herein comprises Dulbecco's Modified Essential Media (also abbreviated DMEM herein). Particularly preferred is DMEM-low glucose (also DMEM-LG herein) (Invitrogen, Carlsbad, Calif.). The DMEM-low glucose is preferably supplemented with 15% (v/v) fetal bovine serum (e.g. defined fetal bovine serum, Hyclone, Logan Utah), antibiotics/antimycotics (preferably penicillin (100 Units/milliliter), streptomycin (100 milligrams/milliliter), and amphotericin B (0.25 micrograms/milliliter), (Invitrogen, Carlsbad, Calif.)), and 0.001% (v/v) 2-mercaptoethanol (Sigma, St. Louis Mo.). In some cases different growth media are used, or different supplementations are provided, and these are normally indicated in the text as supplementations to Growth Medium. Also relating to the present invention, the term standard growth conditions, as used herein refers to culturing of cells at 37.degree. C., in a standard atmosphere comprising 5% CO.sub.2. Relative humidity is maintained at about 100%. While foregoing the conditions are useful for culturing, it is to be understood that such conditions are capable of being varied by the skilled artisan who will appreciate the options available in the art for culturing cells, for example, varying the temperature, CO.sub.2, relative humidity, oxygen, growth medium, and the like.

Presently preferred are methods which provide cells which require no exogenous growth factors, except as are available in the supplemental serum provided with the Growth Medium. Also provided herein are methods of deriving umbilical cells capable of expansion in the absence of particular growth factors. The methods are similar to the method above, however they require that the particular growth factors (for which the cells have no requirement) be absent in the culture medium in which the cells are ultimately resuspended and grown in. In this sense, the method is selective for those cells capable of division in the absence of the particular growth factors. Preferred cells in some embodiments are capable of growth and expansion in chemically-defined growth media with no serum added. In such cases, the cells may require certain growth factors, which can be added to the medium to support and sustain the cells. Presently preferred factors to be added for growth on serum-free media include one or more of FGF, EGF, IGF, and PDGF. In more preferred embodiments, two, three or all four of the factors are add to serum free or chemically defined media. In other embodiments, LIF is added to serum-free medium to support or improve growth of the cells.

Also provided are methods wherein the cells can expand in the presence of from about 5% to about 20% oxygen in their atmosphere. Methods to obtain cells that require L-valine require that cells be cultured in the presence of L-valine. After a cell is obtained, its need for L-valine can be tested and confirmed by growing on D-valine containing medium that lacks the L-isomer.

Methods are provided wherein the cells can undergo at least 25, 30, 35, or 40 doublings prior to reaching a senescent state. Methods for deriving cells capable of doubling to reach 10.sup.14 cells or more are provided. Preferred are those methods which derive cells that can double sufficiently to produce at least about 10.sup.14, 10.sup.15, 10.sup.16, or 10.sup.17 or more cells when seeded at from about 10.sup.3 to about 10.sup.6 cells/cm.sup.2 in culture. Preferably these cell numbers are produced within 80, 70, or 60 days or less. In one embodiment, cord tissue MSC cells are isolated and expanded, and possess one or more markers selected from a group comprising of CD10, CD13, CD44, CD73, CD90, CD141, PDGFr-alpha, or HLA-A,B,C. In addition, the cells do not produce one or more of CD31, CD34, CD45, CD117, CD141, or HLA-DR, DP, DQ.

In order to determine the quality of MSC cultures, flow cytometry is performed on all cultures for surface expression of SH-2, SH-3, SH-4 MSC markers and lack of contaminating CD14- and CD-45 positive cells. Cells were detached with 0.05% trypsin-EDTA, washed with DPBS+2% bovine albumin, fixed in 1% paraformaldehyde, blocked in 10% serum, incubated separately with primary SH-2, SH-3 and SH-4 antibodies followed by PE-conjugated anti-mouse IgG(H+L) antibody. Confluent MSCs in 175 cm² flasks are washed with Tyrode's salt solution, incubated with medium 199 (M199) for 60 min, and detached with 0.05% trypsin-EDTA (Gibco). Cells from 10 flasks were detached at a time and MSCs were resuspended in 40 ml of M199+1% human serum albumin (HSA; American Red Cross, Washington D.C., USA). MSCs harvested from each 10-flask set were stored for up to 4 h at 4° C. and combined at the end of the harvest. A total of 2-10′10⁶ MSCs/kg were resuspended in M199+1% HSA and centrifuged at 460 g for 10 min at 20° C. Cell pellets were resuspended in fresh M199+1% HSA media and centrifuged at 460 g for 10 min at 20° C. for three additional times. Total harvest time was 2-4 h based on MSC yield per flask and the target dose. Harvested MSCs are cryopreserved in Cryocyte (Baxter, Deerfield, Ill., USA) freezing bags using a rate controlled freezer at a final concentration of 10% DMSO (Research Industries, Salt Lake City, Utah, USA) and 5% HSA. On the day of infusion cryopreserved units were thawed at the bedside in a 37° C. water bath and transferred into 60 ml syringes within 5 min and infused intravenously into patients over 10-15 min. Patients are premedicated with 325-650 mg acetaminophen and 12.5-25 mg of diphenhydramine orally. Blood pressure, pulse, respiratory rate, temperature and oxygen saturation are monitored at the time of infusion and every 15 min thereafter for 3 h followed by every 2 h for 6 h.

In one embodiment, MSCs are generated according to protocols previously utilized for treatment of patients utilizing bone marrow derived MSCs. Specifically, bone marrow is aspirated (10-30 ml) under local anesthesia (with or without sedation) from the posterior iliac crest, collected into sodium heparin containing tubes and transferred to a Good Manufacturing Practices (GMP) clean room. Bone marrow cells are washed with a washing solution such as Dulbecco's phosphate-buffered saline (DPBS), RPMI, or PBS supplemented with autologous patient plasma and layered on to 25 ml of Percoll (1.073 g/ml) at a concentration of approximately 1-2′10⁷ cells/ml. Subsequently the cells are centrifuged at 900 g for approximately 30 min or a time period sufficient to achieve separation of mononuclear cells from debris and erythrocytes. Said cells are then washed with PBS and plated at a density of approximately 1′10⁶ cells per ml in 175 cm² tissue culture flasks in DMEM with 10% FCS with flasks subsequently being loaded with a minimum of 30 million bone marrow mononuclear cells. The MSCs are allowed to adhere for 72 h followed by media changes every 3-4 days. Adherent cells are removed with 0.05% trypsin-EDTA and replated at a density of 1′10⁶ per 175 cm². Said bone marrow MSC may be administered intravenously, or in a preferred embodiment to a patient suffering from pulmomary fibrosis. Although doses may be determined by one of skill in the art, and are dependent on various patient characteristics, intravenous administration may be performed at concentrations ranging from 1-10 million MSC per kilogram, with a preferred dose of approximately 2-5 million cells per kilogram.

Exosomes, also referred to as “particles” may comprise vesicles or a flattened sphere limited by a lipid bilayer. The particles may comprise diameters of 40-100 nm. The particles may be formed by inward budding of the endosomal membrane. The particles may have a density of .about.1.13-1.19 g/ml and may float on sucrose gradients. The particles may be enriched in cholesterol and sphingomyelin, and lipid raft markers such as GM1, GM3, flotillin and the src protein kinase Lyn. The particles may comprise one or more proteins present in MSC or MSC conditioned medium, such as a protein characteristic or specific to the MSC or MSC conditioned media. They may comprise RNA, for example miRNA. Said particles may possess one or more genes or gene products found in MSC or medium which is conditioned by culture of MSC. The particle may comprise molecules secreted by the MSC. Such a particle, and combinations of any of the molecules comprised therein, including in particular proteins or polypeptides, may be used to supplement the activity of, or in place of, the MSC or medium conditioned by the MSC for the purpose of for example treating or preventing a disease. Said particle may comprise a cytosolic protein found in cytoskeleton e.g. tubulin, actin and actin-binding proteins, intracellular membrane fusions and transport e.g. annexins and rab proteins, signal transduction proteins e.g. protein kinases, 14-3-3 and heterotrimeric G proteins, metabolic enzymes e.g. peroxidases, pyruvate and lipid kinases, and enolase-1 and the family of tetraspanins e.g. CD9, CD63, CD81 and CD82. In particular, the particle may comprise one or more tetraspanins. The particles may comprise mRNA and/or microRNA. The particle may be used for any of the therapeutic purposes that the MSC or MSC conditioned media may be put to use.

In some embodiments, MSCs are dedifferentiated prior to administration in order to benefit telomere length. Accordingly, the present disclosure provides a method of isolating a subpopulation of MSCs useful for modulating telomere length comprising: a) providing MSCs that express an Oct-4-reporter; and b) isolating cells positive for the reporter. Definitions from part A that are relevant to this section apply to this section as well. MSCs that express an Oct-4-reporter can be produced by various methods known in the art, including, without limitation, introduction of a nucleic acid construct or vector by transformation, transfection or transduction as herein defined. In one embodiment, the Oct-4-reporter gene is introduced by lentiviral transduction. The term “reprogramming potential” as used herein refers to the potential of the cells to regain progenitor or stem cell capacity or pluripotent state. The term “increased reprogramming potential” as used herein means that the reprogramming potential is greater than the potential for a mixed population of MSCs that have not been selected or isolated. The term “Oct-4-reporter” as used herein refers to DNA sequences that are bound by Oct-4 upstream of a reporter that allow or enhance transcription of the downstream sequences of the reporter. Oct-4 reporters are known in the art. For example, an Oct-4 reporter is described in Hotta et al. 2009 and Okumura-Nakanishi et al. 2005 incorporated herein by reference in its entirety. The term “reporter gene” and “reporter” as used herein refers to any gene that encodes a protein that is identifiable. Reporter genes and reporter products are readily identified by a skilled person. In an embodiment, more than one reporter gene/reporter is used. In one embodiment, the reporter gene comprises a fluorescent protein (such as green fluorescent protein, GFP) and the cells are isolated in step (b) by detection of the fluorescent protein under fluorescence. In another embodiment, the reporter gene encodes a gene conferring antibiotic resistance, such as to puromycin, and the cells are isolated by survival in the presence of the antibiotic. In one embodiment, the MSCs are dermal MSCs. The reporter gene could also encode a tag and the cells can be isolated based on immuno separation (http://www.miltenyibiotec.com/en/PG.sub.--167.sub.--501_MACSelect_Vector-s_and_Tag_Vector_Sets.aspx). The disclosure also provides a method of generating reprogrammed MSC-derived induced pluripotent stem (iPS) cells comprising: a) providing (i) a population of MSCs with increased expression of Oct-4 and (ii) a mixed population of MSCs or a population of Oct-4 negative MSCs; b) treating the MSCs of a) with Oct-4, Sox-2, Nanog and Lin-28; and c) culturing the cells of (b) under conditions that allow the production of iPS cells. In one embodiment, the MSCs in b) are treated with Oct-4, Sox-2, Nanog and Lin-28 by introducing the respective genes by viral transduction, such as lentiviral transduction.

The term “stem cell” as used herein refers to a cell that has the ability for self-renewal. In one embodiment, the stem cell is a pluripotent stem cell. The term “pluripotent” as used herein refers to an undifferentiated cell that maintains the ability to allow differentiation into various cell types. The term “induced pluripotent stem cell” refers to a pluripotent stem cell that has been artificially derived from a non-pluripotent stem cell. The term “Sox-2” as used herein refers to the gene product of the Sox-2 gene and includes Sox-2 from any species or source and includes variants, analogs and fragments or portion of Sox-2 that retain activity. The Sox-2 protein may have any of the known published sequences for Sox-2, which can be obtained from public sources such as GenBank. An example of such a sequence includes, but is not limited to, NM.sub.--003106. The term “Nanog” as used herein refers to the gene product of the Nanog gene and includes Nanog from any species or source and includes variants, analogs and fragments or portion of Nanog that retain activity. The Nanog protein may have any of the known published sequences for Nanog, which can be obtained from public sources such as GenBank. An example of such a sequence includes, but is not limited to, NM.sub.--024865. The term “Lin-28” as used herein refers to the gene product of the Lin-28 gene and includes Lin-28 from any species or source and includes variants, analogs and fragments or portions of Lin-28 that retain activity. The Lin-28 protein may have any of the known published sequences for Lin 28, which can be obtained from public sources such as GenBank. An example of such a sequence includes, but is not limited to, BC028566.2. Lin-28 also called CSDD1 or ZCCHC1 or Lin28A.

Reference to particular buffers, media, reagents, cells, culture conditions and the like, or to some subclass of same, is not intended to be limiting, but should be read to include all such related materials that one of ordinary skill in the art would recognize as being of interest or value in the particular context in which that discussion is presented. For example, it is often possible to substitute one buffer system or culture medium for another, such that a different but known way is used to achieve the same goals as those to which the use of a suggested method, material or composition is directed. In a particularly preferred embodiment MSCs are cultured in the cell culture system which is a cell culture system, comprising a cell culture medium, preferably in a culture vessel, in particular a cell culture medium supplemented with a substance suitable and determined for protecting the cells from in vitro aging and/or inducing in an unspecific or specific reprogramming. In a particularly preferred embodiment an inducing substance according to the present invention is a substance selected from the group consisting of reversin, cord blood serum, lithium, a GSK-3 inhibitor, resveratrol, pterostilbene, selenium, a selenium-containing compound, EGCG ((−)-epigallocatechin-3-gallate), valproic acid and salts of valproic acid, in particular sodium valproate. In one embodiment of the present invention, a concentration of reversin from 0.5 to 10 .mu.M, preferably of 1 .mu.M is added to the MSC culture. In a furthermore preferred embodiment the present invention foresees to use resveratrol in a concentration of 10 to 100 .mu.M, preferably 50 .mu.M. In a furthermore preferred embodiment the present invention foresees to use selenium or a selenium containing compound in a concentration from 0.05 to 0.5 .mu.M, preferably of 0.1 .mu.M. In another embodiment, cord blood serum is added at a concentration of 0.1%-20% volume to the volume of tissue culture media. In furthermore preferred embodiment the present invention foresees to use EGCG in a concentration from 0.001 to 0.1 .mu.M, preferably of 0.01 .mu.M. In a furthermore preferred embodiment the present invention foresees to use valproic acid or sodium valproate in a concentration from 1 to 10 .mu.M, in particular of 5 .mu.M.

Furthermore, in the MSC culture procedure, the cell culture medium comprises, optionally in combination with one or more of the substances specified above, at least one transient proteolysis inhibitor. The use of at least one proteolysis inhibitor in the cell culture medium of the present invention increases the time the reprogramming proteins derived from the mRNA or any endogenous genes will be present in the cells and thus facilitates in an even more improved way the reprogramming by the transfected mRNA derived factors. The present invention uses in a particularly preferred embodiment as a transient proteolysis inhibitor a protease inhibitor, a proteasome inhibitor and/or a lysosome inhibitor. In a particularly preferred embodiment the proteosome inhibitor is selected from the group consisting of MG132, TMC-95A, TS-341 and MG262. In a furthermore preferred embodiment the protease inhibitor is selected from the group consisting of aprotinin, G-64 and leupeptine-hemisulfat. In a furthermore preferred embodiment the lysosomal inhibitor is ammonium chloride. In one embodiment the present invention also foresees a cell culture medium comprising at least one transient inhibitor of mRNA degradation. The use of a transient inhibitor of mRNA degradation increases the half-life of the reprogramming factors as well. Another embodiment of the present invention a condition suitable to allow translation of the transfected reprogramming mRNA molecules in the cells is an oxygen content in the cell culture medium from 0.5 to 21%. More particular, and without wishing to be bound to the theory, oxygen is used to further induce or increase Oct4 by triggering Oct4 via Hif1a, in these situations concentrations of oxygen lower than atmospheric concentration are used, and can be ranging from 0.1% to 10%. In a preferred embodiment conditions that are suitable to support reprogramming of the cells by the mRNA molecules in the cells are selected; more particularly, these conditions require a temperature from 30 to 38.degree. C., preferably from 31 to 37.degree. C., most preferably from 32 to 36.degree. C. The glucose content of the medium is in a preferred embodiment of the present invention below 4.6 g/l, preferably below 4.5 g/l, more preferably below 4 g/l, even more preferably below 3 g/l, particularly preferably below 2 g/I and most preferably it is 1 g/1. DMEM media containing 1 g/l glucose being preferred for the present invention are commercially available as “DMEM low glucose” from companies such as PAA, Omega Scientific, Perbio and Biosera. More particular, and without wishing to be bound to the theory, high glucose conditions adversely support aging of cells (methylation, epigenetics) in vitro which may render the reprogramming difficult. In a furthermore preferred embodiment of the present invention the cell culture medium contains glucose in a concentration from 0.1 g/l to 4.6 g/l, preferably from 0.5 g/l to 4.5 g/l and most preferably from 1 g/l to 4 g/1. In accordance with the invention presented herein, the concept of identifying the “sufficient period of time” to allow stable expression of the at least one gene regulator in absence of the reprogramming agent and the “sufficient period of time” in which the cell is to be maintained in culture conditions supporting the transformation of the desired cell is within the skill of those in the art. The sufficient or proper time period will vary according to various factors, including but not limited to, the particular type and epigenetic status of cells (e.g. the cell of the first type and the desired cell), the amount of starting material (e.g. the number of cells to be transformed), the amount and type of reprogramming agent(s), the gene regulator(s), the culture conditions, presence of compounds that speed up reprogramming (ex, compounds that increase cell cycle turnover, modify the epigenetic status, and/or enhance cell viability), etc. In various embodiments the sufficient period of time to allow a stable expression of the at least one gene regulator in absence of the reprogramming agent is about 1 day, about 2-4 days, about 4-7 days, about 1-2 weeks, about 2-3 weeks or about 3-4 weeks. In various embodiments the sufficient period of time in which the cells are to be maintained in culture conditions supporting the transformation of the desired cell and allow a stable expression of a plurality of secondary genes is about 1 day, about 2-4 days, about 4-7 days, or about 1-2 weeks, about 2-3 weeks, about 3-4 weeks, about 4-6 weeks or about 6-8 weeks. In preferred embodiments, at the end of the transformation period, the number of transformed desired cells is substantially equivalent or even higher than an amount of cells a first type provided at the beginning.

The invention teaches the use of MSC cells, either autologous or allogeneic, for treatment of diseases associated with shortened telomeres. Means of manipulation of MSCs are disclosed, as well as MSCs of different tissue origins, which actively inhibit telomere shortening. In one embodiment of the invention MSCs are selected for ability to inhibit accelerated erosion of telomeric ends and utilized as a cellular therapy for prevention and/or treatment of idiopathic pulmonary fibrosis. In one embodiment MSCs are generated by means known in the art. The route of administration, dosage and frequency will be dependent on the disease process, as well as stage of the disease. In one embodiment allogeneic MSCs are administered in a non-manipulated manner but selected from sources characterized by immune modulatory activity such as placental MSCs. In other embodiments of the invention MSCs are cultured under conditions capable of inducing retrodifferentiation so as to endow an immature phenotype, said immature phenotype correlating with enhanced ability to restore telomere length. For example, MSCs may be cultured in the presence of a histone deacetylase inhibitor such as valproic acid. Conditions of growing cells in HDAC inhibitors to induce dedifferentiation are known in the art and incorporated by reference [32, 33]. Other means of inducing dedifferentiation in addition to HDAC inhibitors may also be utilized in the context of the current invention including 8-Br-cAMP [34], M-CSF treatment [35], exposure to reveresine [36], and exposure to stem cell extracts [37]. Quantification of MSC dedifferentiation can be performed by assessment of extracellular markers, as well as intracellular markers such as SOX-2, NANOG, and OCT-4.

In some embodiments of the invention, cells express markers selected from markers comprising of Telomerase, Nanog, Sox2, .beta.-III-Tubulin, NF-M, MAP2, APP, GLUT, NCAM, NeuroD, Nurr1, GFAP, NG2, Olig1, Alkaline Phosphatase, Vimentin, Osteonectin, Osteoprotegrin, Osterix, Adipsin, Erythropoietin, SM22-.alpha., HGF, c-MET, .alpha.-1-Antriptrypsin, Ceruloplasmin, AFP, PEPCK 1, BDNF, NT-4/5, TrkA, BMP2, BMP4, FGF2, FGF4, PDGF, PGF, TGF.alpha., TGF.beta., and VEGF. Growth of MSCs for immune modulation may be performed in a variety of tissue culture media, for example, in one embodiment of the invention, one medium for the culturing of the cells of the invention in comprises Dulbecco's Modified Essential Media (DMEM). Particularly preferred is DMEM-low glucose (DMEM-LG) (Invitrogen, Carlsbad, Calif.). The DMEM-LG is preferably supplemented with serum, most preferably fetal bovine serum or human serum. Typically, 15% (v/v) fetal bovine serum (e.g. defined fetal bovine serum, Hyclone, Logan Utah) is added, along with antibiotics/antimycotics ((preferably 100 Unit/milliliter penicillin, 100 milligrams/milliliter streptomycin, and 0.25 microgram/milliliter amphotericin B; Invitrogen, Carlsbad, Calif.)), and 0.001% (v/v) 2-mercaptoethanol (Sigma, St. Louis Mo.). In some cases different growth media are used, or different supplementations are provided, and these are normally indicated in the text as supplementations to Growth Medium. In certain chemically-defined media the cells may be grown without serum present at all. In such cases, the cells may require certain growth factors, which can be added to the medium to support and sustain the cells. Presently preferred factors to be added for growth on serum-free media include one or more of bFGF, EGF, IGF-I, and PDGF. In more preferred embodiments, two, three or all four of the factors are add to serum free or chemically defined media. In other embodiments, LIF is added to serum-free medium to support or improve growth of the cells.

In some embodiments, generation of cells for use within the invention calls for dedifferentiation using mRNA transfer. In one embodiment, RNA or mRNA is extracted to achieve pluripotency in the ‘target’ cells include by way of example: Human and/or Mouse Embryonic Stem cell, Human and/or Mouse Primordial Germ Cells, Mouse Teratocarcinoma cells, Mouse Embryonic-carcinoma cells, preimplantation embryos and oocytes from any species including human and vertebrates such as amphibians, fish, and mammals. Examples of recipient or target cells into which RNA or mRNA can be introduced to achieve pluripotency or transdifferentiation in the ‘target’ cells include by way of example primary MSCs. Various sources of MSCs may be used, depending on tissue and age. Examples of somatic cells which may be used as the donor cell for transdifferentiation include any cell type that is desired for cell therapies including by way of example hepatocytes, lymphocytes, beta cells, neural cells, cardiac cells, lung cells. The current invention effects dedifferentiation of target cells using total RNA or mRNA. The mRNA or total RNA used to effect dedifferentiation is preferably isolated from cells that are either pluripotent or which are capable of turning into pluripotent cells (oocyte). Examples thereof include by way of example Ntera cells, human or other ES cells, primordial germ cells, and blastocysts. Alternatively the RNA used to effect dedifferentiation may comprise mRNA encoding specific transcription factors. The total RNA or mRNA's may be delivered into target cells by different methods including e.g., electroporation, liposomes, and mRNA injection. Target cells into which RNA's are introduced and which are to be dedifferentiated according to the invention are cultured in a medium containing one or more constituents that facilitates transformation of cell phenotype. These constituents include by way of example epigenetic modifiers such as DNA demethylating agents, HDAC inhibitors, histone modifiers; and cell cycle manipulation and pluripotent or tissue specific promoting agents such as helper cells which promote growth of pluripotent cells, growth factors, hormones, and bioactive molecules. Examples of DNA methylating agents include 5-azacytidine (5-aza), MNNG, 5-aza, N-methl-N′-nitro-N-nitrosoguanidine, temozolomide, procarbazine, et al. Examples of methylation inhibiting drugs agents include decitabine, 5-azacytidine, hydralazine, procainamide, mitoxantrone, zebularine, 5-fluorodeoxycytidine, 5-fluorocytidine, anti-sense oligonucleotides against DNA methyltransferase, or other inhibitors of enzymes involved in the methylation of DNA. Examples of histone deacetylase (“HDAC”) inhibitor is selected from a group consisting of hydroxamic acids, cyclic peptides, benzamides, short-chain fatty acids, and depudecin. Examples of hydroxamic acids and derivatives of hydroxamic acids include, but are not limited to, trichostatin A (TSA), suberoylanilide hydroxamic acid (SAHA), oxamflatin, suberic bishydroxamic acid (SBHA), m-carboxycinnamic acid bishydroxamic (CBHA), and pyroxamide. Examples of cyclic peptides include, but are not limited to, trapoxin A, apicidin and FR901228. Examples of benzamides include but are not limited to MS-27-275. Examples of short-chain fatty acids include but are not limited to butyrates (e.g., butyric acid and phenylbutyrate (PB)) Other examples include CI-994 (acetyldinaline) and trichostatine. Preferred examples of histone modifiers include PARP, the human enhancer of zeste, valproic acid, and trichostatine. Particular constituents that the inventors utilize in a preferred media in order to facilitate RNA transformation and dedifferentiation of the RNA comprising target cells into pluripotent cells include trichostatine, valproic acid, zebularine and 5-aza. Target cells into which RNA is introduced are cultured for a sufficient time in media that promotes RNA transformation until dedifferentiated cells (pluripotent) cells are obtained. One embodiment of the invention teaches that introduction of total RNA or mRNA's from one cell type such as a pluripotent or somatic cell into a desired human somatic cell such as a MSC in order to dedifferentiate or transdifferentiate such cell into a pluripotent cell or a different somatic cell corresponding to the lineage of the cell from which the donor total RNA is derived. This may be sufficient to effect cell dedifferentiation or transdifferentiation. In some instances this methodology may be combined with other methods and treatments involved in the epigenetic status of the recipient or target cell such as the exposure to DNA and histone demethylating agents, histone deacetylase inhibitors, and/or histone modifiers. This invention therefore describes a method of changing the fate or phenotype of cells. By using epigenetic modifications, the subject methods can dedifferentiate or transdifferentiate cells. This invention is aimed to solve the problem of immuno-rejection which is evident when incompatible cells/tissues are used for transplantation. Cells from one patient can be transformed into a different type of cell allowing for the derivation of cells needed for the treatment of a particular disease the patient is suffering from. One of the types of cells that can be produced by this invention is pluripotent stem cells. This invention also offers an opportunity to the research community to study the mechanisms involved in cell differentiation and disease progression.

In one embodiment, MSC exosomes, or particles may be produced by culturing MSC cells in a medium to condition it. The MSC cells may comprise human umbilical tissue derived cells which possess markers selected from a group comprising of CD90, CD73 and CD105. The medium may comprise DMEM. The DMEM may be such that it does not comprise phenol red. The medium may be supplemented with insulin, transferrin, or selenoprotein (ITS), or any combination thereof. It may comprise FGF2. It may comprise PDGF AB. The concentration of FGF2 may be about 5 ng/ml FGF2. The concentration of PDGF AB may be about 5 ng/ml. The medium may comprise glutamine-penicillin-streptomycin or b-mercaptoethanol, or any combination thereof. The cells may be cultured for about 1, 2, 3, 4, 5, 6, 7, 8, 9, 10 days or more, for example 3 days. The conditioned medium may be obtained by separating the cells from the medium. The conditioned medium may be centrifuged, for example at 500 g. it may be concentrated by filtration through a membrane. The membrane may comprise a >1000 kDa membrame. The conditioned medium may be concentrated about 50 times or more. The conditioned medium may be subject to liquid chromatography such as HPLC. The conditioned medium may be separated by size exclusion. Any size exclusion matrix such as Sepharose may be used. As an example, a TSK Guard column SWXL, 6.times.40 mm or a TSK gel G4000 SWXL, 7.8.times.300 mm may be employed. The eluent buffer may comprise any physiological medium such as saline. It may comprise 20 mM phosphate buffer with 150 mM of NaCl at pH 7.2. The chromatography system may be equilibrated at a flow rate of 0.5 ml/min. The elution mode may be isocratic. UV absorbance at 220 nm may be used to track the progress of elution. Fractions may be examined for dynamic light scattering (DLS) using a quasi-elastic light scattering (QELS) detector. Fractions which are found to exhibit dynamic light scattering may be retained. For example, a fraction which is produced by the general method as described above, and which elutes with a retention time of 11-13 minutes, such as 12 minutes, is found to exhibit dynamic light scattering. The r.sub.h of particles in this peak is about 45-55 nm. Such fractions comprise MSC derived particles such as exosomes.

In one embodiment of the invention, MSC cells, and/or MSC cell derivatives, such as exosomes are administered to patients in which an increase in telomere length is desired, together with agents known in the art to increase telomere length. Guidance is given to one of skill in the art in the practice of the invention by publications in the field, for example, Townsley et al treated 27 patients suffering from accelerated telomere shortening with the synthetic sex hormone danazol orally at a dose of 800 mg per day for a total of 24 months. The goal of treatment was the attenuation of accelerated telomere attrition, and the primary efficacy end point was a 20% reduction in the annual rate of telomere attrition measured at 24 months. The occurrence of toxic effects of treatment was the primary safety end point. Hematologic response to treatment at various time points was the secondary efficacy end point. Telomere attrition as part of the disease pathology was reduced in all 12 patients who could be evaluated for the primary end point; in the intention-to-treat analysis, 12 of 27 patients (44%; 95% confidence interval [CI], 26 to 64) met the primary efficacy end point. Unexpectedly, almost all the patients (11 of 12, 92%) had a gain in telomere length at 24 months as compared with baseline (mean increase, 386 bp [95% CI, 178 to 593]); in exploratory analyses, similar increases were observed at 6 months (16 of 21 patients; mean increase, 175 bp [95% CI, 79 to 271]) and 12 months (16 of 18 patients; mean increase, 360 bp [95% CI, 209 to 512]). Hematologic responses occurred in 19 of 24 patients (79%) who could be evaluated at 3 months and in 10 of 12 patients (83%) who could be evaluated at 24 months. Known adverse effects of danazol—elevated liver-enzyme levels and muscle cramps—of grade 2 or less occurred in 41% and 33% of the patients, respectively [38].

The assessment of telomere length, which is important for the practice of the invention, may be assessed by using means widely known in the art and incorporated by reference [39-42].

In one embodiment of the invention, MSCs are treated with one or more “Inhibitor(s) of DNA methylation”. This term refers to an agent that can inhibit DNA methylation. DNA methylation inhibitors have demonstrated the ability to restore suppressed gene expression. Suitable agents for inhibiting DNA methylation include, but are not limited to 5-azacytidine, 5-aza-2-deoxycytidine, 1-.beta.-D-arabinofuranosil-5-azacytosine, and dihydro-5-azacytidine, and zebularine (ZEB), BIX (histone lysine methyltransferase inhibitor), and RG108. Concentration of DNA methylation inhibitors, as well as duration of exposure, is dependent on ability to induce expansion of plasticity. For example, expansion of plasticity may be measured by ability of MSCs to differentiate into other tissues. In a preferred embodiment, MSCs are utilized to differentiate into chondrocytes. Methods of differentiating MSCs in chondrocytes (or in some situations mesenchymal stems cells into chondrocytes), and assessment of differentiation are disclosed in the following publications [43-45].

“Inhibitor of histone deacetylation” refers to an agent that prevents the removal of the acetyl groups from the lysine residues of histones that would otherwise lead to the formation of a condensed and transcriptionally silenced chromatin. Histone deacetylase inhibitors fall into several groups, including: (1) hydroxamic acids such as trichostatin (A) [46-49], (2) cyclic tetrapeptides, (3) benzamides, (4) electrophilic ketones, and (5) aliphatic acid group of compounds such as phenylbutyrate and valporic acid. Suitable agents to inhibit histone deacetylation include, but are not limited to, valporic acid (VPA) [50-61], phenylbutyrate and Trichostatin A (TSA). One example, in the area of mesenchymal stem cells, of valproic acid enhancing pluripotency and therapeutic properties is provided by Killer et al. who showed that culture of cells with valproic acid enhanced immune regulatory and metabolic properties of mesenchymal stem cells. The culture systems described, as well as means of assessment, are provided to allow one of skill in the art to have a starting point for the practice of the current invention [62, 63]. Without being bound to theory, valproic acid in the context of the current invention may be useful to increasing in vitro proliferation of dedifferentiated MSCs while preventing senescence associated stress. For example, Zhai et al showed that in an in vitro pre-mature senescence model, valproic acid treatment increased cell proliferation and inhibited apoptosis through the suppression of the p16/p21 pathway. In addition, valproic acid also inhibited the G2/M phase blockage derived from the senescence stress [64]. In some embodiments of the invention, small RNAs that act as small activating RNA (saRNA) which induce activation of OCT4 expression are applied to MSCs to induce dedifferentiation. In some cases this is combined with histone deacetylase inhibitors and/or GSK3 inhibitors and/or DNA methyltransferase inhibitors, in order to induce a dedifferentiated phenotype in the MSCs. Small RNAs that act as small activating RNAs of the OCT4 promotor are described in the following publications [65-70].

### Example 1: Lithium Enhances Ability of JadiCells to Increase T Regulatory Cell Numbers in Mice

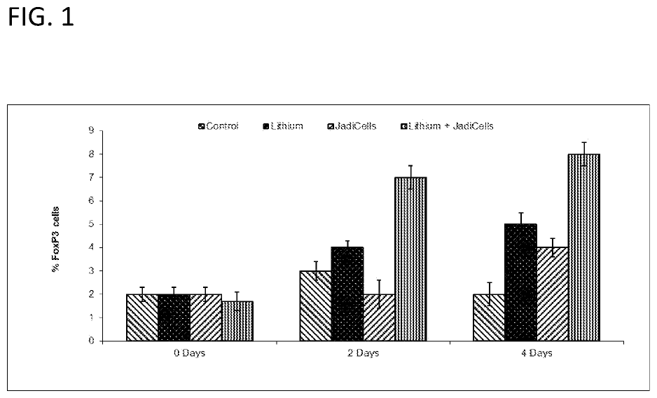

10 week old Female BALB/c mice were administered 150 mg/kg of lithium chloride daily by the IP route. Additionally some animals received Jadicells at 500,000. Quantification of T regulatory cells was performed by intracellular staining for FoxP3 expression. Results are shown in FIG. 1.

### Example 2: JadiCells are Superior to BM and Adipose MSC

Bleomycin (BLM) administered intratracheally 3 mg/kg in female 10 week old C57/BL6 mice

JadiCellst™ or BM-MSC or adipose administered by tail vein at 5 days after BLM

Animals sacrificed at day 12 and assessed for fibrosis (hydroxyproline content)

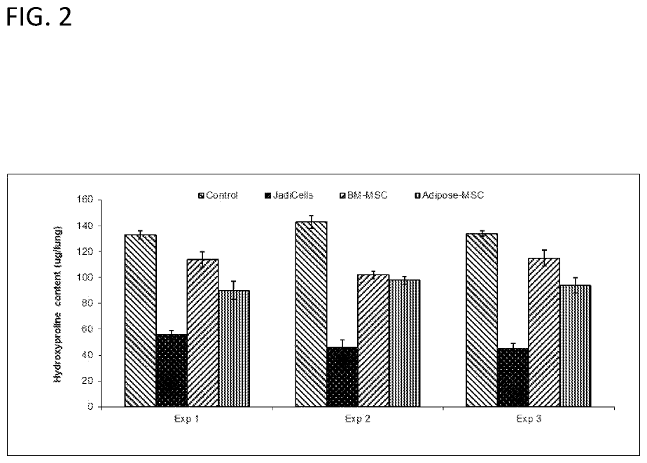

Results are shown in FIG. 2

### Example 3: Lithium Chloride Enhances Antifibrotic Activities of JadiCells

Bleomycin (BLM) administered intratracheally 3 mg/kg in female 10 week old C57/BL6 mice

JadiCellst™ was injected by tail vein at 5 days after BLM. Lithium was injected IP as described in example 1.

Animals sacrificed at day 12 and assessed for fibrosis (hydroxyproline content).

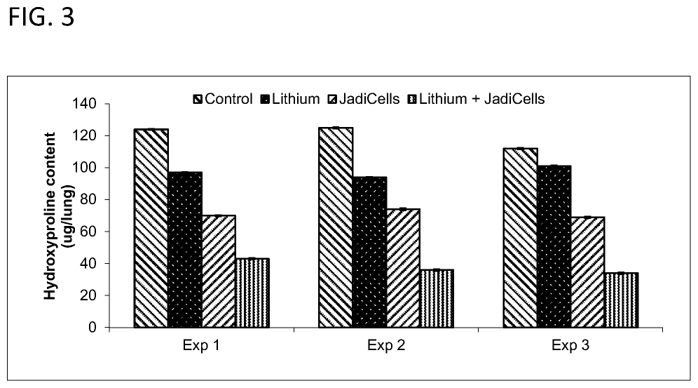

Results are shown in FIG. 3.

### REFERENCES

1. de Wilde, A. H., et al., Host Factors in Coronavirus Replication. Curr Top Microbiol Immunol, 2018. 419: p. 1-42.
2. Raj, V. S., et al., Dipeptidyl peptidase 4 is a functional receptor for the emerging human coronavirus-EMC. Nature, 2013. 495(7440): p. 251-4.
3. Xia, S., et al., Inhibition of SARS-CoV-2 (previously 2019-nCoV) infection by a highly potent pan-coronavirus fusion inhibitor targeting its spike protein that harbors a high capacity to mediate membrane fusion. Cell Res, 2020.
4. Sevajol, M., et al., Insights into RNA synthesis, capping, and proofreading mechanisms of SARS-coronavirus. Virus Res, 2014. 194: p. 90-9.
5. Zou, X., et al., Single-cell RNA-seq data analysis on the receptor ACE2 expression reveals the potential risk of different human organs vulnerable to 2019-nCoV infection. Front Med, 2020.
6. Wang, D., et al., Clinical Characteristics of 138 Hospitalized Patients With 2019 Novel Coronavirus-Infected Pneumonia in Wuhan, China. JAMA, 2020.
7. Channappanavar, R. and S. Perlman, Pathogenic human coronavirus infections: causes and consequences of cytokine storm and immunopathology. Semin Immunopathol, 2017. 39(5): p. 529-539.
8. Bellani, G., et al., Epidemiology, Patterns of Care, and Mortality for Patients With Acute Respiratory Distress Syndrome in Intensive Care Units in 50 Countries. JAMA, 2016. 315(8): p. 788-800.
9. Yan, X., et al., Nrf2/Keap1/ARE Signaling Mediated an Antioxidative Protection of Human Placental Mesenchymal Stem Cells of Fetal Origin in Alveolar Epithelial Cells. Oxid Med Cell Longev, 2019. 2019: p. 2654910.
10. Cui, P., et al., Human amnion-derived mesenchymal stem cells alleviate lung injury induced by white smoke inhalation in rats. Stem Cell Res Ther, 2018. 9(1): p. 101.
11. Zhang, S., et al., Nrf2 transfection enhances the efficacy of human amniotic mesenchymal stem cells to repair lung injury induced by lipopolysaccharide. J Cell Biochem, 2018. 119(2): p. 1627-1636.
12. Zhao, R., et al., Serious adverse events of cell therapy for respiratory diseases: a systematic review and meta-analysis. Oncotarget, 2017. 8(18): p. 30511-30523.
13. Cheng, S. L., C. H. Lin, and C. L. Yao, Mesenchymal Stem Cell Administration in Patients with Chronic Obstructive Pulmonary Disease: State of the Science. Stem Cells Int, 2017. 2017: p. 8916570.
14. Cronkhite, J. T., et al., Telomere shortening in familial and sporadic pulmonary fibrosis. Am J Respir Crit Care Med, 2008. 178(7): p. 729-37.
15. Alder, J. K., et al., Short telomeres are a risk factor for idiopathic pulmonary fibrosis. Proc Natl Acad Sci USA, 2008. 105(35): p. 13051-6.
16. Diaz de Leon, A., et al., Telomere lengths, pulmonary fibrosis and telomerase (TERT) mutations. PLoS One, 2010. 5(5): p. e10680.
17. Alder, J. K., et al., Telomere length is a determinant of emphysema susceptibility. Am J Respir Crit Care Med, 2011. 184(8): p. 904-12.
18. Tsang, A. R., et al., hTERT mutations associated with idiopathic pulmonary fibrosis affect telomerase activity, telomere length, and cell growth by distinct mechanisms. Aging Cell, 2012. 11(3): p. 482-90.
19. Faner, R., et al., Abnormal lung aging in chronic obstructive pulmonary disease and idiopathic pulmonary fibrosis. Am J Respir Crit Care Med, 2012. 186(4): p. 306-13.
20. Fernandez, B. A., et al., A Newfoundland cohort of familial and sporadic idiopathic pulmonary fibrosis patients: clinical and genetic features. Respir Res, 2012. 13: p. 64.
21. Armanios, M., Telomeres and age-related disease: how telomere biology informs clinical paradigms. J Clin Invest, 2013. 123(3): p. 996-1002.
22. Codd, V., et al., Identification of seven loci affecting mean telomere length and their association with disease. Nat Genet, 2013. 45(4): p. 422-7, 427e1-2.
23. Haver, V. G., et al., Telomere length and outcomes in ischaemic heart failure: data from the COntrolled ROsuvastatin multiNAtional Trial in Heart Failure (CORONA). Eur J Heart Fail, 2015. 17(3): p. 313-9.
24. Alba, A. C., et al., Changes in circulating progenitor cells are associated with outcome in heart failure patients: a longitudinal study. Can J Cardiol, 2013. 29(12): p. 1657-64.
25. Chu, K., et al., Circulating endothelial progenitor cells as a new marker of endothelial dysfunction or repair in acute stroke. Stroke, 2008. 39(5): p. 1441-7.
26. Sobrino, T., et al., The increase of circulating endothelial progenitor cells after acute ischemic stroke is associated with good outcome. Stroke, 2007. 38(10): p. 2759-64.
27. Vemparala, K., et al., Early accelerated senescence of circulating endothelial progenitor cells in premature coronary artery disease patients in a developing country—a case control study. BMC Cardiovasc Disord, 2013. 13: p. 104.
28. Olivieri, F., et al., Cellular senescence in cardiovascular diseases: potential age-related mechanisms and implications for treatment. Curr Pharm Des, 2013. 19(9): p. 1710-9.
29. Satoh, M., et al., Association between oxidative DNA damage and telomere shortening in circulating endothelial progenitor cells obtained from metabolic syndrome patients with coronary artery disease. Atherosclerosis, 2008. 198(2): p. 347-53.
30. Kushner, E. J., et al., Aging and endothelial progenitor cell telomere length in healthy men. Clin Chem Lab Med, 2009. 47(1): p. 47-50.
31. Fyhrquist, F., O. Saijonmaa, and T. Strandberg, The roles of senescence and telomere shortening in cardiovascular disease. Nat Rev Cardiol, 2013. 10(5): p. 274-83.
32. Moon, J. H., et al., Induction of neural stem cell-like cells (NSCLCs) from mouse astrocytes by Bmi1. Biochem Biophys Res Commun, 2008. 371(2): p. 267-72.
33. Huang, Y., et al., Histone deacetylase inhibitor significantly improved the cloning efficiency of porcine somatic cell nuclear transfer embryos. Cell Reprogram, 2011. 13(6): p. 513-20.
34. Wang, Y. and J. Adjaye, A cyclic AMP analog, 8-Br-cAMP, enhances the induction of pluripotency in human fibroblast cells. Stem Cell Rev, 2011. 7(2): p. 331-41.
35. Li, Y., R. B. Jalili, and A. Ghahary, Accelerating skin wound healing by M-CSF through generating SSEA-1 and -3 stem cells in the injured sites. Sci Rep, 2016. 6: p. 28979.
36. Li, X., et al., Reversine Increases the Plasticity of Long-Term Cryopreserved Fibroblasts to Multipotent Progenitor Cells through Activation of Oct4. Int J Biol Sci, 2016. 12(1): p. 53-62.
37. Xiong, X. R., et al., Cellular extract facilitates nuclear reprogramming by altering DNA methylation and pluripotency gene expression. Cell Reprogram, 2014. 16(3): p. 215-22.
38. Townsley, D. M., et al., Danazol Treatment for Telomere Diseases. N Engl J Med, 2016. 374(20): p. 1922-31.
39. Baljevic, M., et al., Telomere Length Recovery: A Strong Predictor of Overall Survival in Acute Promyelocytic Leukemia. Acta Haematol, 2016. 136(4): p. 210-218.
40. Barden, A., et al., n-3 Fatty Acid Supplementation and Leukocyte Telomere Length in Patients with Chronic Kidney Disease. Nutrients, 2016. 8(3): p. 175.
41. Harris, S. E., et al., Longitudinal telomere length shortening and cognitive and physical decline in later life: The Lothian Birth Cohorts 1936 and 1921. Mech Ageing Dev, 2016. 154: p. 43-8.
42. Gu, Y., et al., Telomere length, genetic variants and risk of squamous cell carcinoma of the head and neck in Southeast Chinese. Sci Rep, 2016. 6: p. 20675.
43. Mao, Y., et al., Cell type-specific extracellular matrix guided the differentiation of human mesenchymal stem cells in 3D polymeric scaffolds. J Mater Sci Mater Med, 2017. 28(7): p. 100.
44. Pao, S. I., et al., Effect of microgravity on the mesenchymal stem cell characteristics of limbal fibroblasts. J Chin Med Assoc, 2017. 80(9): p. 595-607.
45. Suchorska, W. M., et al., Gene expression profile in human induced pluripotent stem cells: Chondrogenic differentiation in vitro, part A. Mol Med Rep, 2017. 15(5): p. 2387-2401.
46. Ghanjati, F. and S. Santourlidis, Effect on Multipotency and Phenotypic Transition of Unrestricted Somatic Stem Cells from Human Umbilical Cord Blood after Treatment with Epigenetic Agents. Stem Cells Int, 2016. 2016: p. 7643218.
47. Dai, X., et al., Somatic nucleus reprogramming is significantly improved by m-carboxycinnamic acid bishydroxamide, a histone deacetylase inhibitor. J Biol Chem, 2010. 285(40): p. 31002-10.
48. Durcova-Hills, G., et al., Reprogramming primordial germ cells into pluripotent stem cells. PLoS One, 2008. 3(10): p. e3531.
49. Huh, Y. H., J. H. Ryu, and J. S. Chun, Regulation of type II collagen expression by histone deacetylase in articular chondrocytes. J Biol Chem, 2007. 282(23): p. 17123-31.
50. Wang, Z., et al., Infusion of Valproic Acid Into the Renal Medulla Activates Stem Cell Population and Attenuates Salt-Sensitive Hypertension in Dahl S Rats. Cell Physiol Biochem, 2017. 42(3): p. 1264-1273.
51. Lim, J., et al., Valproic acid enforces the priming effect of sphingosine-1 phosphate on human mesenchymal stem cells. Int J Mol Med, 2017. 40(3): p. 739-747.
52. Luo, Z., et al., Effects of histone deacetylase inhibitors on regenerative cell responses in human dental pulp cells. Int Endod J, 2017.
53. Hawkins, K. E., et al., Human Amniocytes Are Receptive to Chemically Induced Reprogramming to Pluripotency. Mol Ther, 2017. 25(2): p. 427-442.
54. Mahapatra, P. S., et al., Valproic acid assisted reprogramming of fibroblasts for generation of pluripotent stem cells in buffalo (Bubalus bubalis). Int J Dev Biol, 2017. 61(1-2): p. 81-88.
55. Dziadosz, M., et al., Effects of Pharmacological Agents on Human Amniotic Fluid-Derived Stem Cells in Culture. Stem Cells Dev, 2016.
56. Dehghan, S., et al., Oct4 transcription factor in conjunction with valproic acid accelerates myelin repair in demyelinated optic chiasm in mice. Neuroscience, 2016. 318: p. 178-89.
57. Petkov, S., et al., Long-Term Culture of Porcine Induced Pluripotent Stem-Like Cells Under Feeder-Free Conditions in the Presence of Histone Deacetylase Inhibitors. Stem Cells Dev, 2016. 25(5): p. 386-94.
58. Chen, X., et al., Valproic Acid Enhances iPSC Induction From Human Bone Marrow-Derived Cells Through the Suppression of Reprogramming-Induced Senescence. J Cell Physiol, 2016. 231(8): p. 1719-27.
59. Shi, H., et al., Roles of p53 and ASF1A in the Reprogramming of Sheep Kidney Cells to Pluripotent Cells. Cell Reprogram, 2015. 17(6): p. 441-52.
60. Gajzer, D., et al., Epigenetic and molecular signatures of cord blood CD34(+) cells treated with histone deacetylase inhibitors. Vox Sang, 2016. 110(1): p. 79-89.
61. Dehghan, S., et al., Exogenous Oct4 in combination with valproic acid increased neural progenitor markers: an approach for enhancing the repair potential of the brain. Life Sci, 2015. 122: p. 108-15.
62. Killer, M. C., et al., Immunosuppressive capacity of mesenchymal stem cells correlates with metabolic activity and can be enhanced by valproic acid. Stem Cell Res Ther, 2017. 8(1): p. 100.
63. Fila-Danilow, A., et al., The influence of TSA and VPA on the in vitro differentiation of bone marrow mesenchymal stem cells into neuronal lineage cells: Gene expression studies. Postepy Hig Med Dosw (Online), 2017. 71(0): p. 236-242.
64. Zhai, Y., et al., Histone deacetylase inhibitor valproic acid promotes the induction of pluripotency in mouse fibroblasts by suppressing reprogramming-induced senescence stress. Exp Cell Res, 2015. 337(1): p. 61-7.
65. Wang, J., et al., Identification of small activating RNAs that enhance endogenous OCT4 expression in human mesenchymal stem cells. Stem Cells Dev, 2015. 24(3): p. 345-53.
66. Wang, B. and Y. Hu, Promoter-Targeted Small Activating RNAs Alter Nucleosome Positioning. Adv Exp Med Biol, 2017. 983: p. 53-61.
67. Xu, C., et al., Long non-coding RNA GAS5 controls human embryonic stem cell self-renewal by maintaining NODAL signalling. Nat Commun, 2016. 7: p. 13287.
68. Hadjimichael, C., et al., MicroRNAs for Fine-Tuning of Mouse Embryonic Stem Cell Fate Decision through Regulation of TGF-beta Signaling. Stem Cell Reports, 2016. 6(3): p. 292-301.
69. Li, H. L., et al., miR-302 regulates pluripotency, teratoma formation and differentiation in stem cells via an AKT1/OCT4-dependent manner. Cell Death Dis, 2016. 7: p. e2078.
70. Sandmaier, S. E. and B. P. Telugu, MicroRNA-Mediated Reprogramming of Somatic Cells into Induced Pluripotent Stem Cells. Methods Mol Biol, 2015. 1330: p. 29-36.

1. A method of treating an individual diagnosed with, suspected of having, or preventing in an individual at risk for developing, pulmonary fibrosis comprising administration of a therapeutically effective amount of a lithium salt alone or together with a regenerative cell population.

2. The method of claim 1, wherein said regenerative cell population are mesenchymal stem cells, or stem cell-like cells derived from a source of tissues selected from the group consisting of: a) dermal; b) placental; c) hair follicle; d) deciduous tooth; e) omentum; f) placenta; g) Wharton's jelly; h) bone marrow; i) adipose tissue; j) amniotic membrane; k) amniotic fluid; and l) peripheral blood.

3. The method of claim 2, wherein said peripheral blood is mobilized to enhance concentration of stem cells

4. The method of claim 3, wherein said mobilization is achieved by treatment of said patient with an agent selected from the group consisting of: a) G-CSF; b) M-CSF; c) GM-CSF; d) Mozibil; and e) flt-3 ligand.

5. The method of claim 1, wherein said stem cell is allogeneic to the recipient.

6. The method of claim 1, wherein said pulmonary fibrobis is caused by factors selected from the group consisting of: a) cytokine storm; b) immunological cell infiltration; c) bacterial infection; d) viral infection; e) systemic inflammatory response syndrome; f) systemic inflammation; g) acute radiation syndrome; and h) sepsis.

7. The method of claim 1, wherein said regenerative cell population is administered intravenously.

8. The method of claim 1, wherein said regenerative cell population is administered intranasally.

9. The method of claim 1, wherein said regenerative cell population is administered intratracheally.

10. The method of claim 1, wherein said regenerative cell population is pre-activated with an agent capable of enhancing MSC therapeutic activity.

11. The method of claim 10, wherein said regenerative cell therapeutic activity is selected from the group consisting of: a) mobility towards a chemotactic agent; b) production of anti-inflammatory agents; and c) production of anti-apoptotic agents.

12. The method of claim 11, wherein said mobility towards a chemotactic agent is mediated by enhanced expression of a receptor associated with enhanced chemotaxis.

13. The method of claim 12, wherein said receptor associated with enhanced chemotaxis is CXCR4.

14. The method of claim 11, wherein said anti-inflammatory agents are selected from the group consistig of: a) IL-4; b) IL-10; c) IL-13; d) IL-20; e) IL-27; f) IL-35; g) PGE-2; h) indolamine 2,3 deoxygenase; i) TGF-beta; and J) EGF.

15. The method of claim 1, wherein said regenerative cell population is modified to express enhanced levels of therapeutic cytokines.

16. The method of claim 15, wherein said therapeutic cytokines are selected from the group consisting of: a) cytokines that inhibit apoptosis; b) cytokines that act as growth factors; and c) cytokines which act as immune modulators/anti-inflammatory agents.

17. The method of claim 16, wherein said cytokines that inhibit apoptosis are selected from the group consisting of: a) EGF; b) VEGF; and c) angiopoietin.

18. The method of claim 16, wherein said cytokines that act as growth factors are selected from the group consisting of: a) HGF; b) FGF-1; c) FGF-2; d) KGF; and e) CTNF.

19. The method of claim 16, wherein said cytokines which act as immune modulators/anti-inflammatory agents are selected from the group consisting of: a) IL-4; b) IL-10; c) IL-13; d) IL-20; e) IL-27; f) IL-35; g) PGE-2; h) indolamine 2,3 deoxygenase; i) TGF-beta; and J) neuroaminidase.

20. A method of reprogramming monocytes in the lung of a patient suffering or having the potential to suffer from ARDS comprising administration of a therapeutically effective amount of stem cells alone or in combination with a lithium salt.
